(19) United States
(12) Patent Application Publication
(10) Pub. No.: US 2005/0014838 A1
(43) Pub. Date: Jan. 20, 2005

(54) Method for treating vasculature degeneration and stimulating glucose

(76) Inventors: Stephen H. Monroe, Memphis, TN (US); Robert M. Moore II, Nesbitt, MS (US)

Correspondence Address: PITTS AND BRITTIAN P C, P O BOX 51295, KNOXVILLE, TN, 37950-1295, US

(21) Appl. No.: 10/879,380

(22) Filed: Jun. 29, 2004

Related U.S. Application Data
(60) Provisional application No. 60/483,779, filed on Jun. 30, 2003.

(51) Int. Cl. A61K 31/557

(52) U.S. Cl. 514/573; 514/613

(57) ABSTRACT

A method for treating vascular degeneration and stimulating glucose uptake in diabetics including the administration to a patient of a combination of a dilator of striated muscle microvasculature and chemical entity that stimulates the uptake of glucose in striated muscle in a pharmaceutically acceptable carrier.

### RELATED APPLICATIONS

This application is a non-provisional application claiming priority on Provisional Application Ser. No. 60/483,779 , filed Jun. 30, 2003, entitled: Method for Treating Vascular Degeneration and Stimulating Glucose Uptake in Diabetics, the entirety of which is incorporated herein by reference.

### FIELD OF INVENTION

This application relates to pharmaceuticals and methods for treating vascular degeneration in diabetic patients.

### BACKGROUND OF INVENTION

A critical complication in both type I and type II diabetic patients is the progressive loss of circulation and glucose uptake in the extremities. This physiological state can result in tissue necrosis of the extremities, loss of limbs, or in extreme cases failure of vital organs.

The invention herein disclosed includes a method whereby the circulation in the striated muscle is increased and glucose uptake is stimulated.

### SUMMARY OF INVENTION

The present invention comprises a combination of drugs that increases the blood flow, in tissue beds normally under-perfused in diabetic patients, with the stimulation of glucose uptake in these tissues. This therapeutic endpoint is achieved by the targeting of two unique targets: 1) triggering microvessel dilation in the striated muscle utilizing either a cannabinoid receptor 1 (CB1) agonist or one of the second messengers of this receptor system resulting from activation of cyclooxygenases; 2) stimulating glucose uptake utilizing an agonist of the beta3-adrenergic (β₃-AR) receptors.

In accordance with one aspect of the present invention, there is provided a method wherein the vasculature of the striated muscle of the limbs is triggered to dilate thus increasing circulation. In accordance with a further aspect of the present invention, advantage is taken of the increased circulation by stimulating the uptake of glucose (other sugars) thereby reducing the circulating load of glucose.

### DETAILED DESCRIPTION OF INVENTION

In one embodiment, dilation of the striated muscle microvasculature of a patient suffering from diabetes is achieved by utilizing either a CB1 agonist or an agonist of the EP2 or EP4 receptors. Agonist of the CB 1 receptor include, but are not limited to, THC, nabilone, synhexyl, HU-21 0, anandamide, aracadonylgylcerole, WIN-55940, and other ligands. Agonist of the prostaglandin receptor include the endogenous ligands such as PGE2 and PI as well as metabolically stable prostaglandin analogs such as misiprostil as well as COX-2 metabolites of anandamide and aracadonylglycerol.

In accordance with one aspect of the present invention there is provided a chemical entity that stimulates the uptake of glucose in striated muscle, specifically an agonist of the β₃-AR or a entity that stimulates the up-regulation of the af9rementioned receptor. To this end, agonist of the β₃-AR such as trecadine, SWR-0342SA, and CL316243 may be utilized to stimulate glucose reuptake. Alternatively, compounds such as diazoxide may be utilized to trigger the upregulation of the β₃-AR thus increasing the effective concentration of the receptor under physiological condition thus utilizing the endogenous concentrations of circulating ligands for the β₃-AR. Either of the aforementioned approaches will increase the levels of glucose transporters (GLUT1IGLUT4) with the subsequent cellular uptake of circulating glucose.

By combining these two components in a new and novel way the present inventors address the two major problems facing diabetics, poor circulation and poor glucose metabolism, both of which lead to serious health complications.

1. A method for the treatment of a patient suffering from diabetes comprising the steps of
providing a combination of a dilator of striated muscle microvasculature and a chemical entity that stimulates the uptake of glucose in striated muscle in a pharmaceutically acceptable carrier,
administering a pharmaceutically effective dose of said combination to a patient in need thereof.

2. The method of claim 1 wherein said dilator comprises a cannabinoid receptor 1 (CB1) agonist or one of the second messengers of this receptor system resulting from activation of cyclooxygenases.

3. The method of claim 2 wherein said agonist of the CB 1 receptor include, THC, nabilone, synhexyl, HU-21 0, anandamide, aracadonylgylcerole, WIN-55940, and like functioning ligands.

4. The method of claim 2 wherein said second messenger comprises an agonist of the prostaglandin receptor including the endogenous ligands PGE2 and PI or metabolically stable prostaglandin analogs including misiprostil, COX-2 metabolites of anandamide and aracadonylglycerol.

5. The method of claim 1 wherein said chemical entity that stimulates the uptake of glucose in striated muscle is an agonist of the beta3-adrenergic (β₃-AR) receptors, including trecadine, SWR-0342SA, and CL316243

6. The method of claim 1 wherein said chemical entity that stimulates the uptake of glucose in striated muscle comprises a chemical entity which triggers the upregulation of β₃-AR thus increasing the effective concentration of the receptor under physiological conditions thus utilizing the endogenous concentrations of circulating ligands for the β₃-AR.

7. The method of claim 6 wherein said chemical entity comprises diazoxide.

8. The method of claim 1 wherein said chemical entity functions to increase the levels of glucose transporters GLUT1, GLUT4 or both these transporters, with the subsequent uptake of circulating glucose.

9. A composition for the treatment of diabetics comprising a combination of a dilator of striated muscle microvasculature and a chemical entity that stimulates the uptake of glucose in striated muscle in a pharmaceutically acceptable carrier.

10. The composition of claim 9 wherein said dilator comprises a cannabinoid receptor 1 (CB1) agonist or one of the second messengers of this receptor system resulting from activation of cyclooxygenases.

11. The composition of claim 10 wherein said agonist of the CB 1 receptor include, THC, nabilone, synhexyl, HU-21 0, anandamide, aracadonylgylcerole, WIN-55940, and like functioning ligands

12. The composition of claim 11 wherein said second messenger comprises an agonist of the prostaglandin receptor including the endogenous ligands PGE2 and PI or metabolically stable prostaglandin analogs including misiprostil, COX-2 metabolites of anandamide and aracadonylglycerol.

13. The composition of claim 9 wherein said chemical entity that stimulates the uptake of glucose in striated muscle is an agonist of the beta3-adrenergic (β₃-AR). receptors, including trecadine, SWR-0342SA, and CL316243

14. The method of claim 9 wherein said chemical entity that stimulates the uptake of glucose in striated muscle comprises a chemical entity which triggers the upregulation of β₃-AR thus increasing the effective concentration of the receptor under physiological conditions thus utilizing the endogenous concentrations of circulating ligands for the β₃-AR.

15. The method of claim 15 wherein said chemical entity comprises diazoxide.

16. The method of claim 9 wherein said chemical entity functions to increase the levels-of glucose transporters GLUT1, GLUT4 or both these transporters, with the subsequent uptake of circulating glucose.
